(19) United States
(12) Patent Application Publication
(10) Pub. No.: US 2025/0339568 A1
(43) Pub. Date: Nov. 6, 2025

(54) PRECURSOR AND RADIOTRACER FOR NEUROENDOCRINE THERANOSTICS

(71) Applicant: ITM Oncologics GmbH, Garching (DE)

(72) Inventors: Frank RÖSCH, Berlin (DE); Marian MECKEL, Freising (DE); Sebastian MARX, Haßfurt (DE); Richard BAUM, Hargesheim (DE)

(21) Appl. No.: 18/557,854

(22) PCT Filed: May 2, 2022
(86) PCT No.: PCT/EP2022/061668

§ 371 Date: Oct. 27, 2023

(30) Foreign Application Priority Data
May 4, 2021 (DE) 10 2021 111 452.7

(51) Int. Cl. A61K 51/08

(52) U.S. Cl. CPC A61K 51/088; A61K 2121/00; A61K 2123/00

(57) ABSTRACT

A precursor designated as DAZTA5-PPA2 for PET/CT diagnosis and nuclear therapy|of SSR active lesions with radioisotopes 68Ga and 177Lu provides improved|affinity, specificity and imaging of small metastases.

### SUMMARY

The present invention pertains to a precursor designated as DAZTA⁵-PPA2 or a salt thereof for radiolabeling and targeting of somatostatin receptor 2 (SSR2) comprising the chelator DAZTA⁵ and therewith conjugated peptide ligand PPA2, wherein
DAZTA⁵=
1,4-bis(carboxymethyl)-6-[methyl-carboxymethyl-amino]-6-[pentanoic acid]-1,4-diazepane or
1,4-bis(carboxymethyl)-6-[bis(carboxymethyl)-amino]-6-[pentanoic acid]-1,4-diazepane;
   and
   PPA2=Cpa-cyclo[DCys-Pal-DAph(Cbm)-Lys-Thr-Cys]DTyr-NH₂ with
   Cpa=4-Chloro-phenylalanine, DAph(Cbm)=D-4-Amino-carbamoyl-phenylalanine and
   Pal=Pyridylalanine.

### BACKGROUND

### Nuclear Diagnostics of Neuroendocrine Tumours

Positron Emission Tomography (PET) combined with Computed Tomography (CT) using Gallium-68 (Ga-68 or ⁶⁸Ga) is today a clinically established nuclear diagnostic technique. The U.S. Food and Drug Administration as well as the European Medicines Agency have approved ⁶⁸Ga-labeled 1,4,7,10-tetraazacyclododecane-1,4,7,10-tetraacetic acid (⁶⁸Ga-DOTA-octreotate or ⁶⁸Ga-DOTA-TATE) and ⁶⁸Ga-DOTA-d-Phe(1)-Tyr(3)-octreotide (⁶⁸Ga-DOTA-TOC) for localization of somatostatin receptor (SSR) positive neuroendocrine tumours (NETs) in adult and paediatric patients (in the US) and for adult patients with indication for well-differentiated gastroenteropancreatic neuroendocrine tumours (GEP-NETs) (in the EU). DOTA-TOC and DOTA-TATE are comprised of the DOTA-chelator conjugated with 8 amino acid cyclic peptides with high affinity for somatostatin receptor 2 (SSR2), for which they act as agonists.

The diagnostic value of PET/CT is determined by sensitivity, specificity and accuracy. Sensitivity measures the proportion of positives that are correctly identified (true-positives divided by the sum of true-positives and false-negatives). Specificity measures the proportion of negatives that are correctly identified (true-negatives divided by the sum of true-negatives and false-positives). Diagnostic accuracy relates to the ability of a test to discriminate between the target condition and health. This discriminative faculty can be quantified by the measures of sensitivity and specificity, target to background ratio or area under the receiver operating characteristic curve (ROC curve).

SSR imaging sensitivity can potentially be enhanced by increasing PET-tracer affinity for the targeted SSR or by widening the binding spectrum to encompass SSR3 and SSR5 in addition to SSR2. The latter approach can yield higher tracer uptake in SSR positive target tissue but may also increase off-target uptake, thus resulting in reduced tumour-to-background ratio and inferior image contrast.

The state of the art reports further somatostatin receptor ligands for PET/CT that yield improved diagnostic accuracy and other advantages, among them SSR agonists such as DOTA-NOC (DOTA-1-Nal(3)-octreotide) having high affinity for SSR2, SSR3 and SSR5 or HA-DOTA-TATE (DOTA-iodo-Tyr³-octreotide).

DOTA-ST8951 (DOTA-(4-amino)-D-Phe-cyclo[Cys-Tyr-D-Trp-Lys-Val-Cys]-Thr-NH₂) has high affinity for SSR2 and SSR5, however, increased liver uptake affects target to background ratio. F-18 labeled SSR ligands such as ¹⁸F-FET-BAG-TOCA are reported to have inferior imaging properties.

SSR Agonists vs. Antagonists

In nuclear diagnostics SSR agonists are complemented by SSR antagonists which address a plurality of binding sites on targeted cells. This is attributable to the fact that the majority of SSRs are present in inactive form and hence only accommodate antagonist binding. Accordingly, compared to SSR2 agonist radiotracers complementary SSR2 antagonist radiotracers such as ⁶⁸Ga-DOTA-JR11 and ⁶⁸Ga-NODAGA-LM3 (JR11=Cpa-cyclo [D-Cys-Aph(Hor)-D-Aph(Cbm)-Lys-Thr-Cys]D-Tyr-NH₂; NODAGA=1,4,7-triazacyclononane,1-glutaric acid-4,7-acetic acid; LM3=Cpa-cyclo[D-Cys-Tyr-D-4-amino-Phe(carbamoyl)-Lys-Thr-Cys]D-Tyr-NH₂) show higher uptake in preclinical and clinical settings even though their SSR2 affinities are not significantly higher. In a head to head comparison ⁶⁸Ga-DOTA-JR11 is superior to ⁶⁸Ga-DOTA-TATE in the detection of liver metastases but much less sensitive for bone metastases. This finding emphasizes the importance of image contrast for PET/CT diagnostics.

In order to improve image contrast i.e. specificity, it is mandatory that the PET/CT tracer has low affinity to off-target tissue and disease unrelated receptors. Widening the binding spectrum to receptor subtypes SSR1, SSR3, SSR4 and SSR5 may increase off-target uptake and reduce specificity and image contrast.

Also, selection of a proper target that is either unique to the respective disease or highly over-expressed largely influences the diagnostic outcome. E.g. the most commonly used PET-tracer is the radiolabeled glucose analogue ¹⁸F-2-Fluoro-2-deoxy-D-glucose (¹⁸F-FDG) which is absorbed by various tissues and in case of non-malignant disease in tissue with systemically increased glucose consumption.

The clinically approved theranostic dyad comprising ⁶⁸Ga-DOTA-TATE and ¹⁷⁷Lu-DOTA-TATE has greatly advanced the treatment of patients afflicted by NETs and epitomizes the benefits of nuclear medicine for combatting cancer. Further research to make available improved theranostic tools for NET patients has revealed significant advantages of radiolabeled SSR2-antagonists over their agonist counterparts, both at the preclinical level and in vivo. SSR2-radioantagonists, unlike radioagonists, are not internalized in target cells by endocytosis. Nevertheless, they have displayed superior pharmacokinetics, combining higher and prolonged retention in SSR2-positive tumour lesions with faster washout from healthy tissues. The latter concerns as well healthy organs physiologically expressing SSR2, such as stomach and pancreas. Studies at the molecular and cellular level have shown that radioantagonists occupy larger SSR2 populations on the membrane of target cells, comprising both active and inactive receptors, whereas agonists bind only to the sub-population of active SSR2s on the cell membrane prior to being internalized.

In recent years several types of SSR2-antagonists have been developed and conjugated with various chelators for complexation of bi- and trivalent radiometals for NET diagnosis and therapy. Particularly DOTA-LM3 (DOTA=1,4,7,10-Tetraazacyclododecane-1,4,7,10-tetra-acetic acid; LM3=H-DPhe-cyclo[DCys-Tyr-DAph(Cbm)-Lys-Thr-Cys]-DTyr-NH₂; DAph(Cbm)4=D-4-amino-carbamoyl-phenylalanine, cf. Scheme 1) shows promise for diagnosis and staging of NETs (cf. R. P. Baum, J. Zhang, C. Schuchardt, D. Mueller, H. Maecke; First-in-human study of novel SSTR antagonist ¹⁷⁷Lu-DOTA-LM3 for peptide receptor radionuclide therapy in patients with metastatic neuroendocrine neoplasms: dosimetry, safety and efficacy; Journal of Nuclear Medicine March 2021, jnumed.120.258889; DOI: https://doi.org/10.2967/jnumed.120. 258889).

### Chelators for Complexing Metallic Radioisotopes

According to current knowledge in the art:
the chelator and radioisotope greatly influence affinity and pharmacokinetics of SSR radio-tracers;
DOTA can severely affect SSR ligand affinity;
chelator, radioisotope and SSR ligand interact unpredictably in synergistic or antagonistic manner.

The chelator DOTA, for example, is not well suited for complexing the relatively small (radio) metal Gallium and necessitates elevated reaction temperature which is detrimental for many antibodies and heat-sensitive biomolecules. After complexation ⁶⁸Ga-DOTA chelates require time for cooling prior to intravenous injection, thereby imposing limitations for clinical use due to the short ⁶⁸Ga half-life of 67.7 min.

EP 2 801 582 A1 (para. 102, 129; Table 12) discloses a radiolabeling precursor having structure DOTA-Cpa-cyclo[DCys-Pal-DAph(Cbm)-Lys-Thr-Cys]DTyr-NH₂ which apparently serves as reference example without quantifiable uptake in HEK293-SSR2 tumour cells.

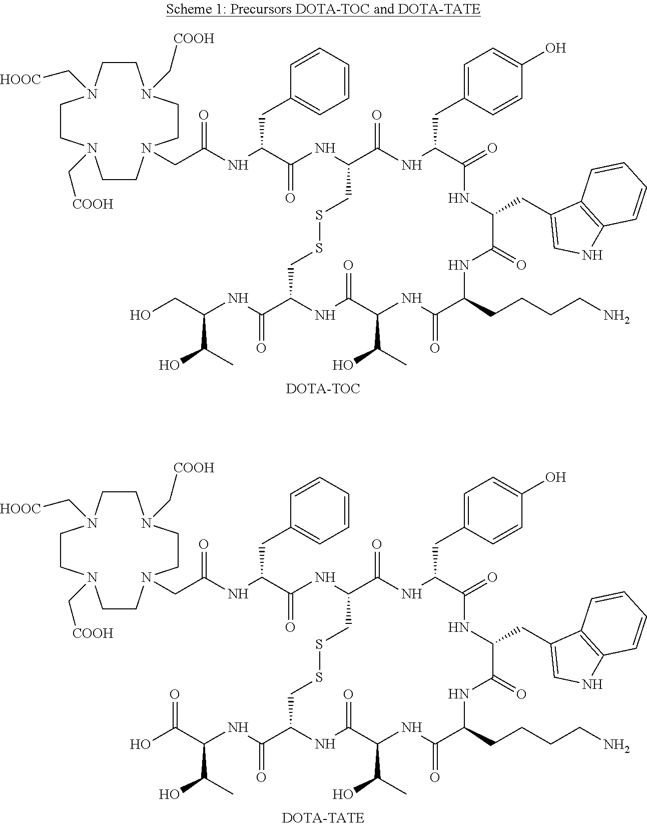 [SMILES: C[C@@H](O)[C@@H]1NC(=O)[C@H](CCCCN)NC(=O)[C@@H](Cc2c[nH]c3ccccc23)NC(=O)[C@@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](CO)[C@@H](C)O)NC1=O ; C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

### DATA as “Hybrid” Chelator

Recently developed chelators of the DATA-type (cf. Scheme 2) exhibit cyclic, acyclic and inter-mediate properties and have advantageous properties for ⁶⁸Ga-labeling compared to established chelators. In particular, they afford rapid quantitative radio labeling with ⁶⁸Ga at ambient temperature in a wide pH range. Furthermore, ⁶⁸Ga-DATA chelates are immune against trans-chelation (DTPA and apo-transferrin) and trans-metalation (Fe^{III}).

Beneath Scheme 2 shows the inventive DAZTA⁵ chelator with the core diazepane ring (1,4-bis(carboxymethyl)-6-[methyl-carboxymethyl-amino]-1,4-diazepane respectively 1,4-bis(carboxymethyl)-6-[bis(carboxymethyl)-amino]-1,4-diazepane).

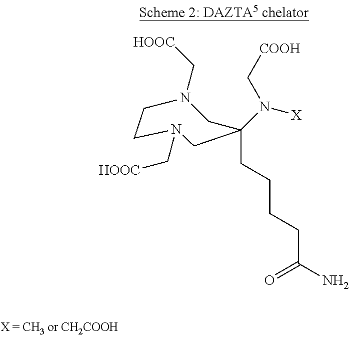

### DETAILED DESCRIPTION

The invention has the object to improve nuclear theranostics of diseases, in particular neuro-endocrine cancer, that are characterized by elevated somatostatin receptor (SSR) expression.

This object is achieved by a precursor designated as DAZTA⁵-PPA2 and having structure

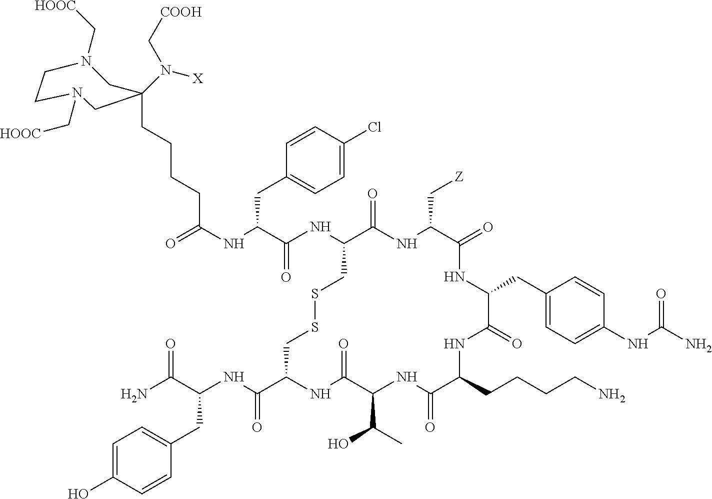

with

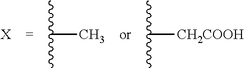

and

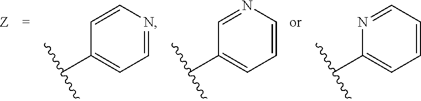

or a salt thereof.

Expedient embodiments of the inventive precursor DAZTA⁵-PPA2 are characterized in that:

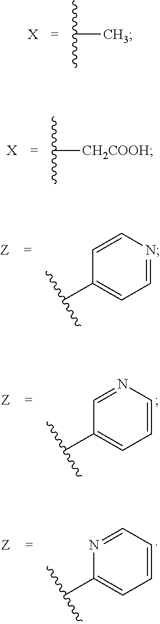

The invention has the further object to provide a radiopharmaceutical for nuclear imaging of diseases associated with elevated SSR expression, in particular neuroendocrine cancer. This object is achieved by radiotracer ⁶⁸Ga-DAZTA⁵-PPA2 consisting of precursor DAZTA⁵-PPA2 with

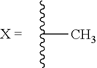

and therewith complexed radioisotope ⁶⁸Ga.

The invention has the further object to provide a radiopharmaceutical for nuclear therapy of diseases associated with elevated SSR expression, in particular neuroendocrine cancer. This object is achieved by radiotracer ¹⁷⁷Lu-DAZTA⁵-PPA2 consisting of precursor DAZTA⁵-PPA2 with

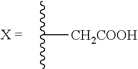

and therewith complexed radioisotope ¹⁷⁷Lu.

Further expedient embodiments of the invention pertain to:
a radiopharmaceutical kit comprising precursor DAZTA⁵-PPA2 with

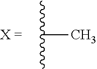

or a salt thereof;
a radiopharmaceutical kit comprising precursor DAZTA⁵-PPA2 with

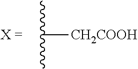

or a salt thereof;
a radiopharmaceutical kit comprising precursor DAZTA⁵-PPA2 with

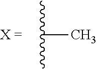

or a salt thereof and a solvent selected from water, 0.45% aqueous NaCl solution, 0.9% aqueous NaCl solution, Ringer solution (Ringer lactate), 5% aqueous dextrose solution and aqueous alcohol solution;
a radiopharmaceutical kit comprising precursor DAZTA⁵-PPA2 with

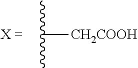

or a salt thereof and a solvent selected from water, 0.45% aqueous NaCl solution, 0.9% aqueous NaCl solution, Ringer solution (Ringer lactate), 5% aqueous dextrose solution and aqueous alcohol solution;
a radiopharmaceutical kit comprising
   a first vial containing precursor DAZTA⁵-PPA2 with X= [symbol] —CH₃ or a salt thereof, and
   a second vial containing precursor DAZTA⁵-PPA2 with X= [symbol] —CH₂COOH or a salt thereof.
a radiopharmaceutical kit comprising
   a first vial containing precursor DAZTA⁵-PPA2 with X= [symbol] —CH₃ or a salt thereof,
   a second vial containing precursor DAZTA⁵-PPA2 with X= [symbol] —CH₂COOH or a salt thereof,
   a third vial containing a solvent selected from water, 0.45% aqueous NaCl solution, 0.9% aqueous NaCl solution, Ringer solution (Ringer lactate), 5% aqueous dextrose solution and aqueous alcohol solution, and
   optionally a fourth vial containing a solvent selected from water, 0.45% aqueous NaCl solution, 0.9% aqueous NaCl solution, Ringer solution (Ringer lactate), 5% aqueous dextrose solution and aqueous alcohol solution.

The invention affords detection of somatostatin receptor expression via ⁶⁸Ga-PET/CT in cases where PET/CT imaging with ⁶⁸Ga-DOTA-TOC or ⁶⁸Ga-DOTA-TATE provides low standardized uptake value (SUV) or difficult to interpret results despite clinical indication for somatostatin receptor positive neuroendocrine tumours.

Precursor DAZTA⁵-PPA2 with X═CH₃ or X═CH₂COOH may be complexed with radioisotope ⁶⁸Ga or ⁴⁴Sc for diagnostic use or with ¹⁷⁷Lu, ⁹⁰Y or ¹⁶¹Tb for therapeutic use. The corresponding radiotracers designated as ⁶⁸Ga-DAZTA⁵-PPA2, ⁴⁴Sc-DAZTA⁵-PPA2, ¹⁷⁷Lu-DAZTA⁵-PPA2, ⁹⁰Y-DAZTA⁵-PPA2 and ¹⁶¹Tb-DAZTA⁵-PPA2 exhibit exceptional target to background ratio i.e. preferential uptake in tumour lesions and low uptake in healthy tissue, particularly liver and spleen tissue. Hence, the inventive radiotracers provide high image contrast, sensitivity and selectivity for diagnosis and treatment of diseases associated with elevated somatostatin receptor expression.

Accordingly, the invention encompasses the following radiotracers:
⁶⁸Ga-DAZTA⁵-PPA2 (X═CH₃), i.e. ⁶⁸Ga-DATA⁵m-PPA2;
⁴⁴Sc-DAZTA⁵-PPA2 (X═CH₃), i.e. ⁴⁴Sc-DATA⁵m-PPA2;
⁶⁸Ga-DAZTA⁵-PPA2 (X═CH₂COOH), i.e. ⁶⁸Ga-AAZTA-PPA2;
⁴⁴Sc-DAZTA⁵-PPA2 (X═CH₂COOH), i.e. ⁴⁴Sc-AAZTA-PPA2;
¹⁷⁷Lu-DAZTA⁵-PPA2 (X═CH₂COOH), i.e. ¹⁷⁷Lu-AAZTA-PPA2;
⁹⁰Y-DAZTA⁵-PPA2 (X═CH₂COOH), i.e. ⁹⁰Y-AAZTA-PPA2;
¹¹¹In-DAZTA⁵-PPA2 (X═CH₂COOH), i.e. ¹¹¹In-AAZTA-PPA2;
¹⁶¹Tb-DAZTA⁵-PPA2 (X═CH₂COOH), i.e. ¹⁶¹Tb-AAZTA-PPA2; and
²²⁵Ac-DAZTA⁵-PPA2 (X═CH₂COOH), i.e. ²²⁵Ac-AAZTA-PPA2.

DAZTA⁵-PPA2 may be readily provided in freeze-dried form and packaged as point-of-use kit with adjuvants such as pH-buffer, antioxidant radical scavengers to prevent radiolysis and lyophilisation bulking agents. Kits containing DAZTA⁵-PPA2 with X═CH₃ or X═CH₂COOH may be used to prepare inventive radiotracers ⁶⁸Ga-DAZTA⁵-PPA2, ⁴⁴Sc-DAZTA⁵-PPA2 or ¹⁷⁷Ga-DAZTA⁵-PPA2 by adding European Pharmacopoeia compliant hydrochloric acid solution containing ⁶⁸GaCl₃, ⁴⁴ScCl₃ or ¹⁷⁷LuCl₃, respectively, at room temperature by simply shaking the reagent mixture. Automated modules with heating compartments are not required.

### EXAMPLES

### Synthesis Strategy

The tert-butyl-protected and carboxylated DAZTA⁵-PPA2 prochelator is synthesized as described beneath in context with Scheme 4 and 5.

The SSR2 peptide ligand PPA2 shown in Scheme 3 is prepared by common solid phase peptide synthesis (SPPS) using Fmoc as protecting group in conjunction with deprotection/coupling cycles (Scheme 6) and purified by reversed-phase chromatography followed by HPLC and MS characterization.

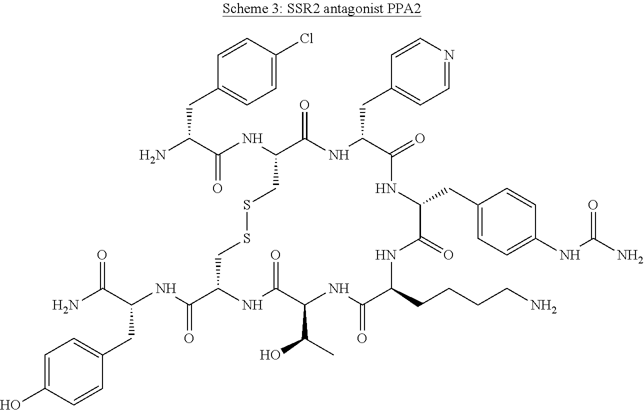 [SMILES: C[C@@H](O)[C@@H]1NC(=O)[C@H](CCCCN)NC(=O)[C@@H](Cc2ccc(NC(N)=O)cc2)NC(=O)[C@@H](Cc2ccncc2)NC(=O)[C@@H](NC(=O)[C@H](N)Cc2ccc(Cl)cc2)CSSC[C@@H](C(=O)N[C@H](Cc2ccc(O)cc2)C(N)=O)NC1=O]

### Reagents and Analysis

Reagents were purchased from Sigma-Aldrich® or Merck® and used without further purification. Purite® water is filtered through a Millex® Millipore filter membrane (0.54 μm). Reaction progress is monitored using silica TLC-plates (silica 60 F254 4.5×4.5 cm, Merck) and UV-absorbance at wavelength 254 nm and/or KMnO₄ titration. Column chromatography is performed with silica gel 60 (Fisher Scientific®, 0.04-0.063 nm).

The chemical identity of synthesized compounds is confirmed by ¹H-, ¹³C-NMR and HRMS except for the DAZTA⁵-PPA2 conjugate, which is characterised by HPLC and HRMS. ¹H-, ¹³C-NMR and HRMS data are stated in S.I. units.

NMR spectra (¹H, ¹³C, HSQC, HMBC) are recorded on an Avance III HD 400 spectrometer (Bruker, United States). Chemical shifts are given in ppm. MS (ESI) is performed with a Thermo Quest Navigator Instrument (Thermo Electron). Mass spectrometry results are given as m/z in g/mol. HPLC is performed with a metal-free Dionex ICS-5000 system equipped with quaternary pump, AS-50 auto sampler, UV/Vis detector and automated fraction collector AFC-3000.

DAZTA⁵ (X═CH₃) Prochelator Synthesis
5-(1,4-Dibenzyl-6-nitro-[1, 4]diazepan-6-yl)-pentanoic acid methyl ester (1)

2-Nitrocyclohexanone (0.608 g, 4.3 mmol) is added to Amberlyst A21 (1.216 g, 2 mass equivalents) in EtOH and stirred for 2 h at 60° C. under argon. N,N′-Dibenzyl-ethylenediamine (1.020 g, 4.3 mmol) and paraformaldehyde (0.446 g, 14.9 mmol) were added and the reaction stirred at 60° C. overnight. The mixture is filtered through Celite®, and solvent removed under reduced pressure. The resulting residue is re-dissolved in CHCl₃ (40 mL) and washed successively with aqueous K₂CO₃ solution (2×30 mL, 0.1 M) and H₂O (30 mL), dried over MgSO₄, filtered and solvent removed under reduced pressure. Purification by silica gel column chromatography (DCM) afforded the title compound as a yellow oil (1.607 g, 85%). Rf=0.80 (DCM).

5-(1,4-Dibenzyl-6-nitro-[1,4]diazepan-6-yl)-pentanoic acid methyl ester (2)

A catalytic amount of Pd(OH)₂/C and acetic acid (50 μL, 0.87 mmol) is added to the protected triamine 1 (0.10 g, 0.29 mmol) in MeOH (20 mL), and the mixture agitated under an atmosphere of hydrogen for 3 h (1 atm H₂). TLC (DCM) is used to confirm complete reduction of the nitro group and cleavage of the benzyl N-substituents. Pd(OH)₂/C is removed using a Celite® filter. The solvent is removed under reduced pressure to afford a yellow oil (0.065 g, 97%).

5-[1,4-Bis-tert-butoxycarbonylmethyl-6-(tert-butoxycarbonylmethyl-amino)-[1,4]diazepan-6-yl]-pentanoic acid methyl ester (3)

tert-Butyl-bromoacetate (0.567 g, 2.91 mmol) is added to 2 (0.208 g, 0.91 mmol) and K₂CO₃ (0.377 g, 2.73 mmol) in MeCN (25 mL), and the mixture stirred for 24 h at 368 K under argon atmosphere. The reaction is monitored by TLC (hexane/ethyl acetate; 1:1) for formation of the tetraalkylated derivative. The solvent is removed under reduced pressure, and the resulting oil re-dissolved in CHCl₃ (25 mL) and washed successively with aqueous K₂CO₃ solution (2×25 mL, 0.1 M) and H₂O (25 mL), dried over MgSO₄, filtered and the solvent removed under reduced pressure. Purification by silica gel column chromatography (hexane/ethyl acetate, 2:1→1:1) affords a yellow oil (0.229 g, 44%). Rf=0.35 (hexane/ethyl acetate; 2:1).

5-[1,4-Bis-tert-butoxycarbonylmethyl-6-(tert-butoxycarbonylmethyl-methyl-amino)-[1,4]diazepan-6-yl]-pentanoic acid methyl ester (4)

Iodomethane (0.023 g, 0.16 mmol) is added to 3 (0.104 g, 0.18 mmol) and K₂CO₃ (0.025 g, 0.18 mmol) in DCM/MeCN (3:1) cooled in an ice-bath. The reaction mixture is allowed to warm to room temperature and left overnight. The solvent is removed under reduced pressure and the resulting oil re-dissolved in CHCl₃ (20 mL), filtered and washed successively with aqueous K₂CO₃ solution (2×20 mL, 0.1 M) and H₂O (20 mL), dried over MgSO₄, filtered and solvent removed under reduced pressure. Purification by silica gel column chromatography (hexane/ethyl acetate, 3:1→2:1) afforded a yellow oil (0.043 g, 46%). Rf=0.38 (hexane/ethyl acetate; 2:1).

5-[1,4-Bis-tert-butoxycarbonylmethyl-6-(tert-butoxycarbonylmethyl-methyl-amino)-[1,4]diazepan-6-yl]-pentanoic acid (5)

LiOH (0.009 g, 0.039 mmol) dissolved in H₂O (0.5 mL) is added to 4 (0.010 g, 0.023 mmol) in THF (0.5 mL), and the mixture stirred at 298 K. The reaction is monitored using LC-ESI MS for ester cleavage. Once complete, the solvent is removed by lyophilisation. H₂O (5 mL) is added and removed by lyophilisation and the procedure repeated two times. The resulting solid is washed with ice-cold DCM (0.5 mL), and dried in vacuo to yield a waxy yellow solid (0.009 g, 70%).

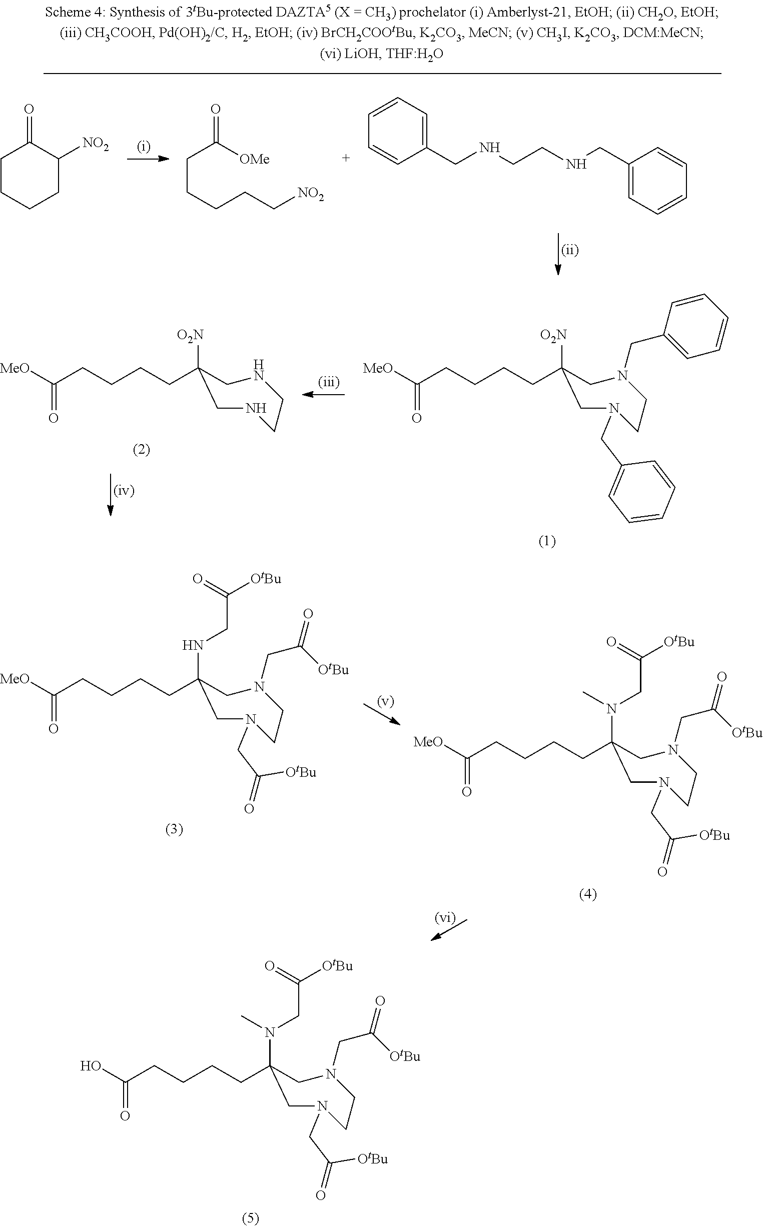

DAZTA⁵ (X═CH₂COOH) Prochelator Synthesis

Prochelator DAZTA⁵ (X═CH₂COOH), commonly also designated as AAZTA may be prepared by a method described by Manzoni et al. (L. Manzoni, L. Belvisi, D. Arosio, M. P. Bartolomeo, A. Bianchi, C. Brioschi, F. Buonsanti, C. Cabella, C. Casagrande, M. Civera, M. De Matteo, L. Fugazza, L. Lattuada, F. Maisano, L. Miragoli, C. Neira, M. Pilkington-Miksa, C. Scolastico; Synthesis of Gd and ⁶⁸Ga Complexes in Conjugation with a Conformationally Optimized RGD Sequence as Potential MRI and PET Tumour-Imaging Probes; ChemMedChem 2012, 7, 1084-1093) as depicted in Scheme 5.

### Compound 6

N,N′-Dibenzylethylenediamine diacetate (14.67 g; 40.7 mmol) is suspended in EtOH (50 mL) and the mixture is heated at 50° C. until a clear solution is obtained. Paraformaldehyde (3.67 g; 122.1 mmol) is added and the suspension is heated at 80° C. for 1.5 h to give a dark orange clear solution. A solution of 6-nitrohexanoic acid methyl ester (R. Ballini, M. Petrini, V. Polzonetti Synthesis 1992, 355-357) (7.13 g; 40.7 mmol) in EtOH (10 mL) is added dropwise. The obtained solution is left to cool to room temperature, stirred for 18 h at room temperature then for 4.5 h at 50° C. The mixture is evaporated, the residue dissolved in EtOAc (100 mL) and the solution washed with aq. Na2CO3 and brine. The aqueous phase is separated and extracted with EtOAc (1×50 ml; 1×30 mL). The organic phases are collected, dried (Na2SO4), filtered and evaporated. The crude is purified by flash chromatography (silica gel column, 90:10 petroleum ether/EtOAc) to give 23 as a pale yellow oil (10.8 g; 24.6 mmol). (60%). ¹H-NMR (CDCl₃, 400 MHZ): δ 0.80 (m, 2H), 1.32 (m, 2H), 1.58 (m, 2H), 2.12 (t, 2H, J=7.5 Hz), 2.62 (m, 4H), 2.96 (d, 2H, J=14.2 Hz), 3.52 (d, 2H, J=14.2 Hz), 3.59 (d, 2H, J=13 Hz), 3.66 (s, 3H), 3.75 (d, 2H, J=13 Hz), 7.28 (m, 10H). ¹³C-NMR (CDCl₃, 100.6 MHz): δ 174.0, 139.5, 129.5, 128.7, 127.6, 95.2, 64.4, 62.0, 59.2, 51.9, 36.9, 33.9, 25.0, 23.0. MS (ESI+) m/z: (M+H⁺), 440.5.

### Compound 7

10% Pd/C (1.5 g) is added to a solution of compound 23 (10 g; 22.8 mmol) in MeOH (400 mL) and the suspension is stirred at 40° C. for 5 h under hydrogen atmosphere. The suspension is filtered (Millipore® filter FT 0.45 μm) and the solution evaporated. The residue is dissolved in MeCN (100 mL) and freshly ground K₂CO₃ (16.8 g; 122 mmol) and Na₂SO₄ (3 g; 21 mmol) are added. t-Butyl bromoacetate (20.8 g; 107 mmol) is added and the orange mixture is stirred and heated at 80° C. for 7 h. The mixture is filtered, more K₂CO₃ (16.8 g; 122 mmol), Na₂SO₄ (3 g; 21 mmol) and t-butyl bromoacetate (0.88 g; 4.5 mmol) is added and the new mixture heated at 80° C. for 9.5 h. The mixture is filtered, evaporated and the residue purified by chromatography (silica gel column, 3:2 n-hexane/EtOAc) to give 24 as a pale yellow oil (7.8 g; 11.4 mmol). (50%). ¹H-NMR (CDCl₃, 400 MHZ): δ 1.46 (s, 36H), 1.62-1.48 (br, 6H), 2.33 (t, 2H, J=7.5 Hz), 2.65 (d, 2H, J=14.2 Hz), 2.83 (m, 4H), 3.00 (d, 2H, J=14.2 Hz), 3.24 (s, 4H), 3.62 (s, 4H), 3.67 (s, 3H). ¹³C-NMR (CDCl₃, 400 MHZ): δ 173.1, 171.2, 81.1, 80.6, 65.5, 63.4, 62.9, 60.8, 52.3, 51.8, 37.6, 34.5, 28.5, 26.1, 22.1. MS (ESI⁺) m/z: (M+H⁺), 686.5, (M+Na⁺), 708.5.

DAZTA⁵ (X═CH₃COOH)/AAZTA (8)

A 1 M solution of LiOH (95.4 mL; 95.4 mmol) is added dropwise to a solution of compound 24 (8.17 g; 11.9 mmol) in THF (200 mL) cooled to 0° C. The solution is then stirred at room temperature for 28 h. The pH of the solution is brought to pH 7 by addition of AcOH (4 mL). Water (50 mL) is added and the THF evaporated. The aqueous residue is extracted with EtOAc (3×75 mL). The organic phases are collected, dried (Na₂SO₄), filtered and evaporated. The crude is purified by flash chromatography (silica gel column, 3:2 n-hexane/EtOAc) to give 4 as a pale yellow oil (3.76 g; 5.6 mmol). (47%). ¹H-NMR (CDCl₃, 400 MHZ): δ 1.48 (s, 36H), 1.66-1.57 (br, 6H), 2.38 (t, 2H, J=7.5 Hz), 2.79-2.67 (br, 6H), 3.03 (d, 2H, J=14.2 Hz), 3.05 (s, 4H), 3.63 (s, S-4 4H). ¹³C-NMR (CDCl₃, 100.6 MHz): δ 178.8, 173.1, 171.0, 81.3, 80.8, 65.4, 63.3, 62.7, 59.4, 37.4, 34.4, 28.4, 28.3, 22.1. MS (ESI⁺) m/z: (M+H⁺), 672.6.

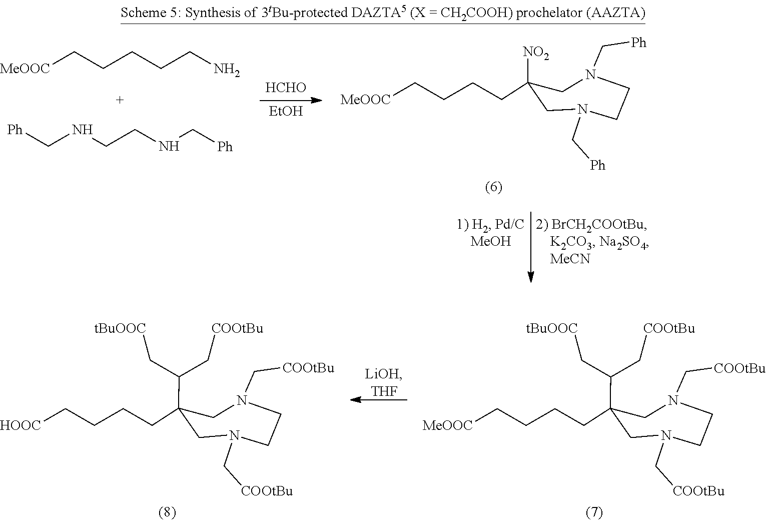

### PPA2 Peptide Synthesis

The PPA2 peptide may be prepared by classical solution synthesis or preferably the established solid-phase technique depicted in Scheme 6 and described in U.S. Pat. Nos. 7,019,109 and 5,874,227, the contents of which are herein incorporated by reference in their entirety. Side-chain protecting groups, which are known in the art, are included as a part of any amino acid that has a particularly reactive side chain, and optionally can be used in the case of others such as Trp, where such amino acids are coupled onto the chain being built upon the resin. Such synthesis provides a fully protected intermediate peptidoresin. Protecting groups are generally split off and the peptide is cleaved from the resin support before oxidizing to create a disulfide bond between the Cys side chains.

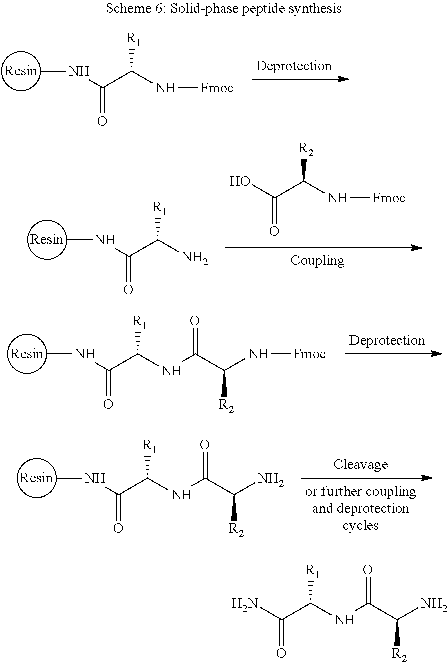

Alternatively, peptide PPA2 may be obtained from various commercial providers such as Peptide Specialty Laboratories GmbH (https://www.peptid.de/).

State of the art PET/CT imaging

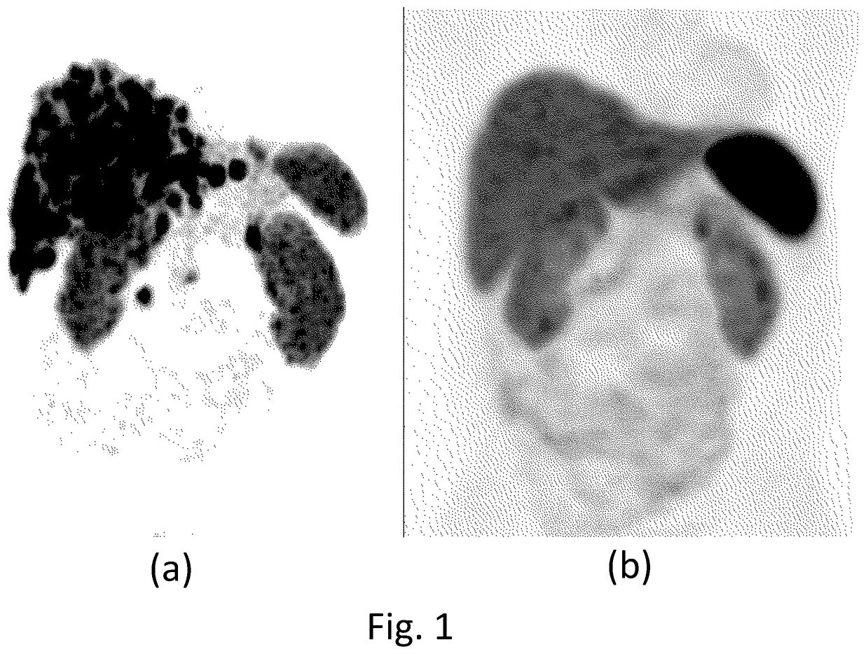

FIG. 1 shows PET/CT images of a patient suffering from hepatic cancer using established radiotracers ⁶⁸Ga-NODAGA-LM3 (FIG. 1a) and ⁶⁸Ga-DOTA-TATE (FIGS. 1b and 1c). ⁶⁸Ga-NODAGA-LM3 provides improved visualization of metastases.

Staging using PET/CT imaging with ⁶⁸Ga-DAZTA⁵-PPA2 (X═CH₃)

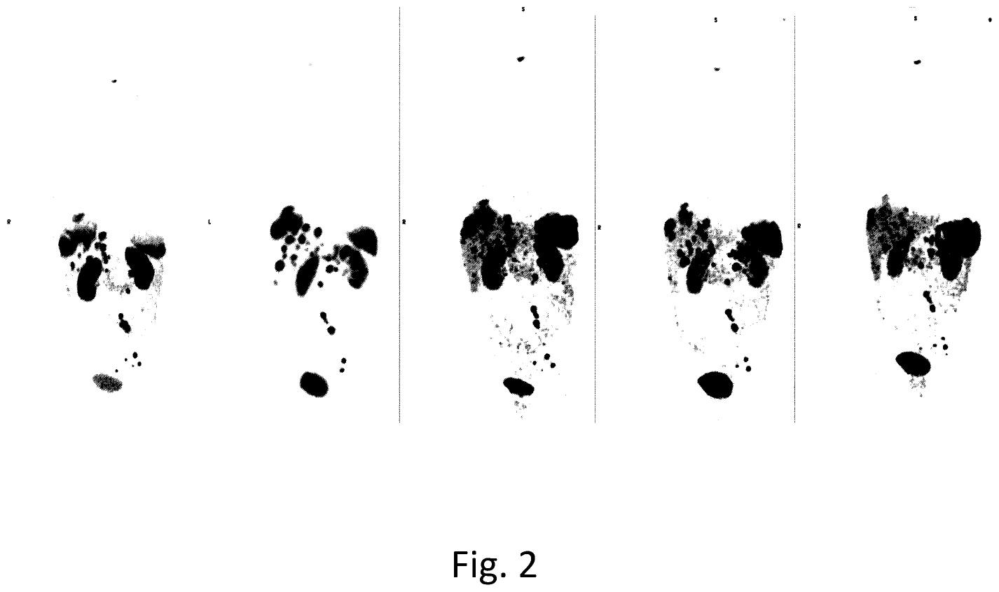

FIG. 2 shows five images of a patient acquired at different times with PET/CT using the inventive radiotracer ⁶⁸Ga-DAZTA⁵-PPA2 with X═CH₃ (i.e. ⁶⁸Ga-DATA⁵m-PPA2) and distinguished by highly sensitive visualization of hepatic metastases, sharp contrast and detection of small metastases and affected lymph nodules.

PET/CT imaging of bone metastases using ⁶⁸Ga-DAZTA⁵-PPA2 (X═CH₃)

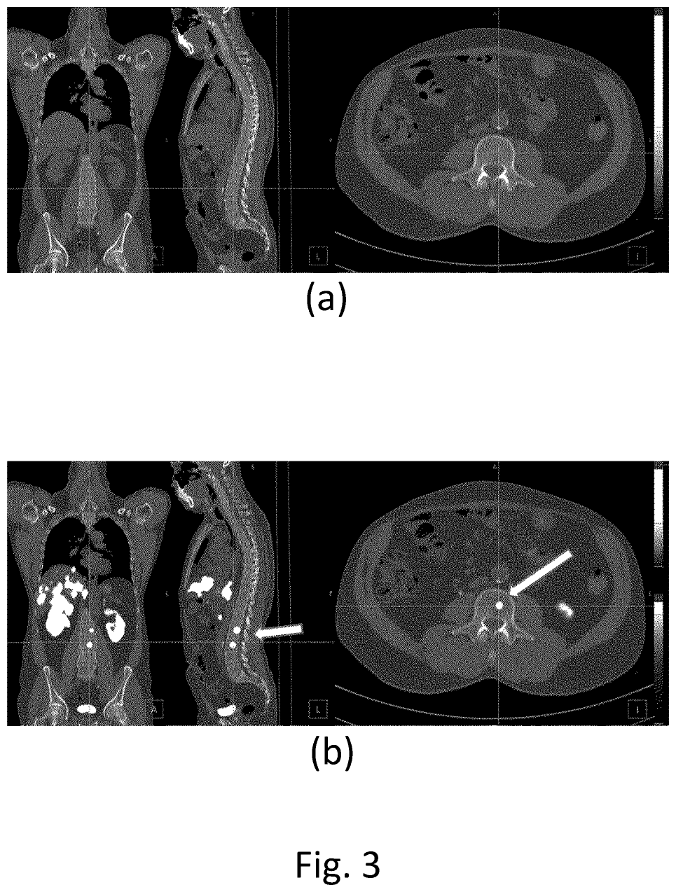

FIG. 3 displays PET/CT images of a patient suffering from multiple bone metastases, not detectable on CT scans as there are no osteoblastic changes. FIGS. 3a and 3b show the CT images and their fusion with PET images, respectively.

PET/CT imaging using ⁶⁸Ga-DAZTA⁵-PPA2 (X═CH₃) of lymph nodes

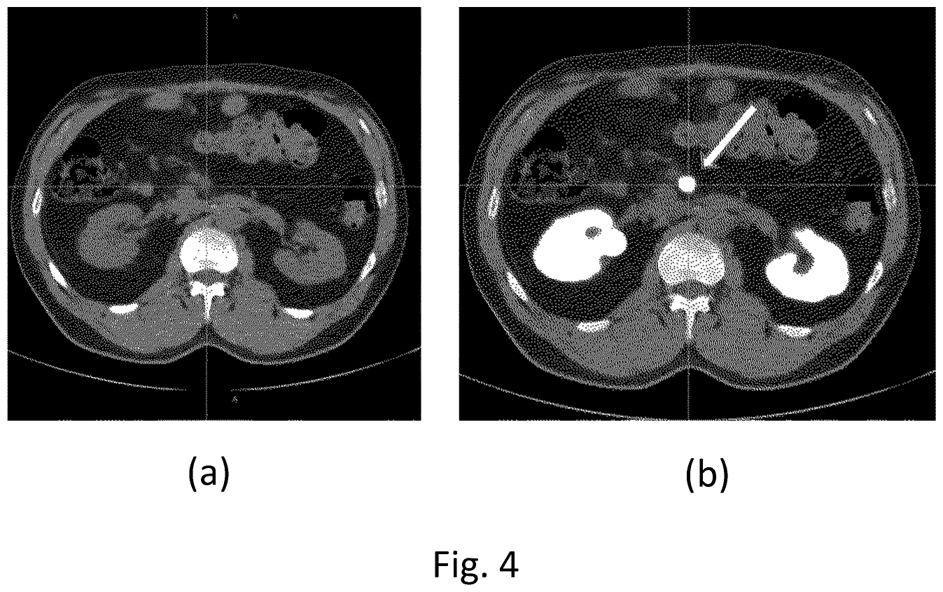

FIG. 4 displays PET/CT images (FIG. 4a) of small abdominal lymph node metastases originating from neuroendocrine cancer with diameter below 6 mm that are not detectable in CT scans (FIG. 4b).

PET/CT imaging using ⁶⁸Ga-NODAGA-LM3 and ⁶⁸Ga-DAZTA⁵-PPA2 (X═CH₃)

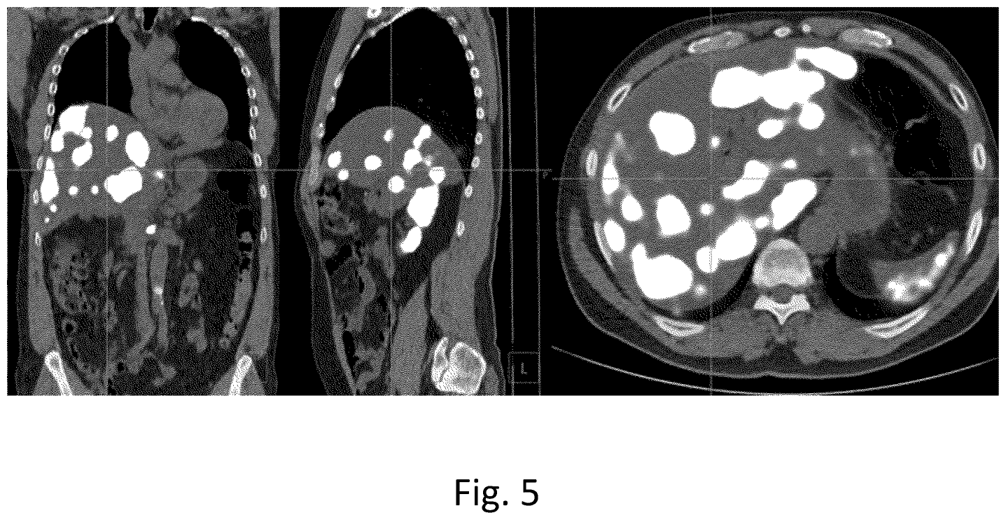

FIG. 5 shows PET/CT images of a patient suffering from hepatic cancer using radiotracers ⁶⁸Ga-NODAGA-LM3 and ⁶⁸Ga-DAZTA⁵-PPA2 with X═CH₃ (i.e. ⁶⁸Ga-DATA^{5m}-PPA2). ⁶⁸Ga-DATA^{5m}-PPA2 provides better visualization of metastases in conjunction with significantly lower background signal from healthy liver and spleen tissue.

PET/CT imaging of breast metastases with ⁶⁸Ga-DAZTA⁵-PPA2 (X═CH₃)

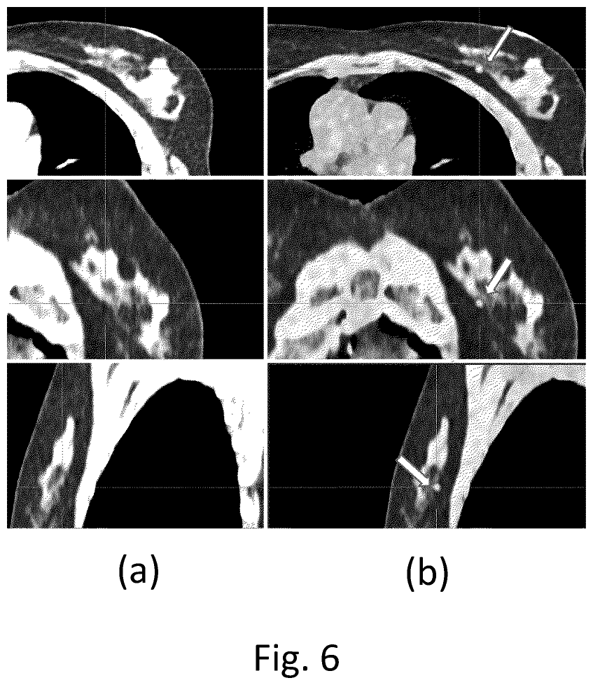

FIG. 6 shows a comparison of images (a) and (b) acquired with regular CT and, respectively PET/CT using ⁶⁸Ga-DAZTA⁵-PPA2 with X═CH₃ (i.e. ⁶⁸Ga-DATA^{5m}-PPA2) of a patient without indication of lesions when examined by magnetic resonance imaging (MRI) and CT. Unlike MRI and CT imaging use of ⁶⁸Ga-DAZTA⁵-PPA2 PET/CT enables detection of metastases having diameters as small as 2 mm.

### Cellular Uptake and Binding

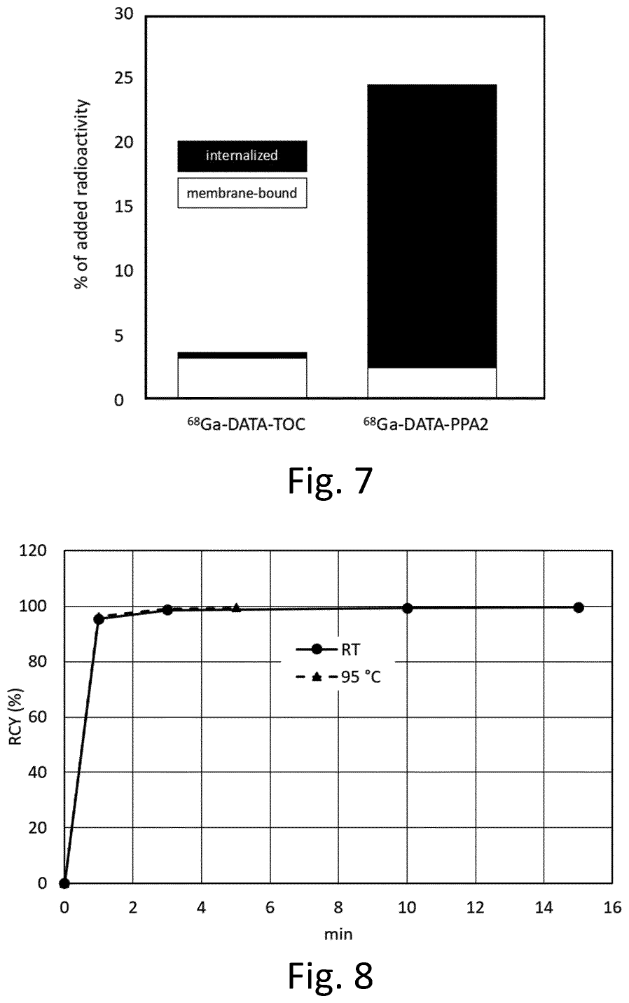

FIG. 7 shows the result of an in vitro cellular uptake comparison of agonist radiotracer ⁶⁸Ga-DATA^{5m}-TOC with antagonist radiotracers ⁶⁸Ga-DAZTA⁵-PPA2 with X═CH₃ (i.e. ⁶⁸Ga-DATA^{5m}-PPA2) using cell line HEK293-SSR2. The inventive radiotracer ⁶⁸Ga-DATA^{5m}-PPA2 exhibits superior overall uptake and a high ratio of membrane binding versus cellular incorporation (endocytosis).

⁶⁸Ga-DAZTA⁵-PPA2 (X═CH₃) radiolabeling kinetics

50 μg of the inventive prochelator DAZTA⁵-PPA2 with X═CH₃ (i.e. DATA^{5m}-PPA2) are added to 500 L sodium acetate buffer (pH 4.5) with therein dissolved ⁶⁸Ga at room temperature (RT) and 95° C. Within 5-10 min radiochemical yields (RCY) in excess of 95% are obtained (cf. FIG. 8).

### In Vitro Stability

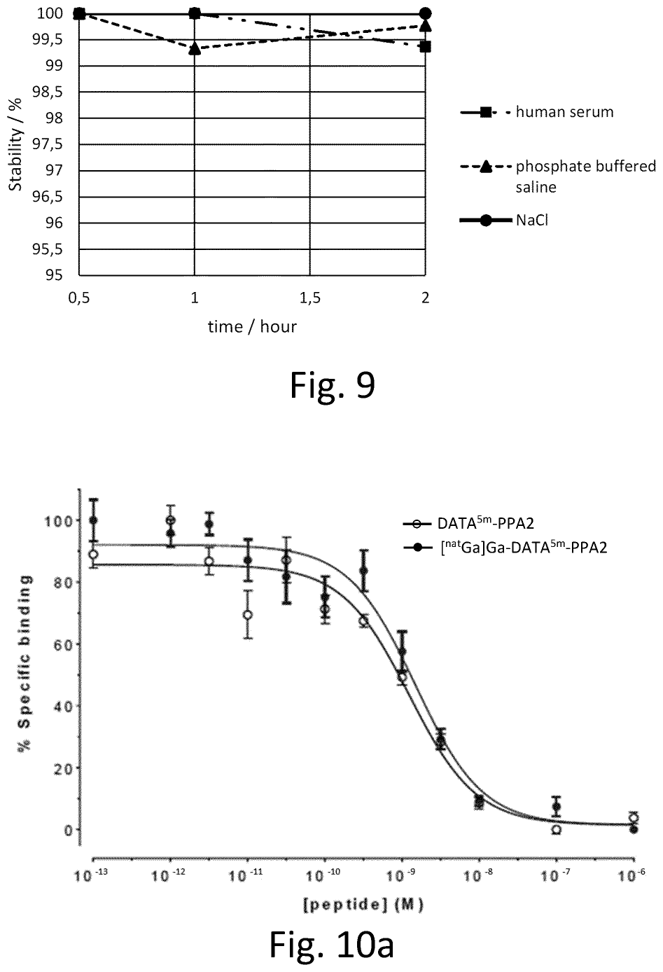

FIG. 9 shows in vitro stability of ⁶⁸Ga-DAZTA⁵-PPA2. The inventive radiotracer DAZTA⁵-PPA2 with X═CH₃ (i.e. DATA^{5m}-PPA2) was suspended in each human serum, phosphate buffered saline (PBS) and physiologic NaCl solution at 37° C. for 120 min. During the 2 h period no measurable degradation could be detected.

### Affinity Assay

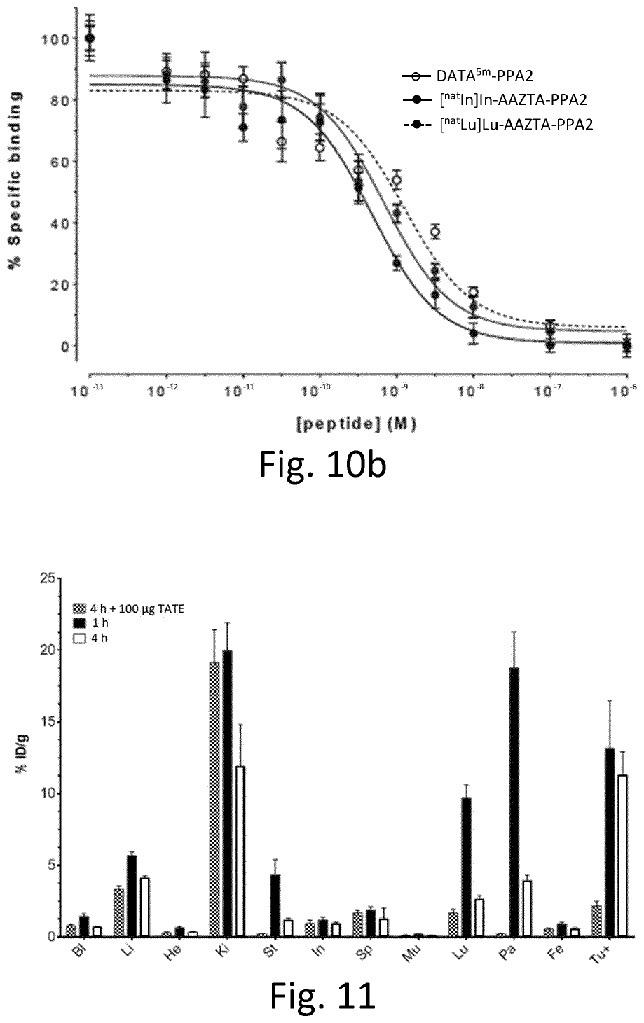

Table 1 depicts relative IC₅₀ values of comparative binding analysis of non-metalated, Ga-, In- and Lu-complexed precursors DATA^{5m}-PPA2 and AAZTA-PPA2 based on displacement assay with [¹²⁵I][Leu⁸, DTrp²², I-Tyr²⁵]SS28 ([¹²⁵I]I-[LTT]SS28) on HEK293-SST2R cell membranes (1 h at 22° C.). FIGS. 10a and 10b show the corresponding measurement curves.

TABLE 1
Relative IC₅₀ values

| Compound | DATA^{5m}-PPA2 | AAZTA-PPA2 |
|---|---|---|
| Non-metalated | 1.24 ± 0.20 | 1.69 ± 0.47 |
| Ga | 1.61 ± 0.32 | n.a. |
| In | n.a. | 0.45 ± 0.05 |
| Lu | n.a. | 0.55 ± 0.37 |

Corresponding P-values for Table 1 data are: P>0.05 for DATA⁵-PPA2 vs. Ga-DATA⁵-PPA2 and In-AAZTA-PPA2 vs. Lu-AAZTA-PPA2; and P<0.01 for AAZTA-PPA2 vs. either In-AAZTA-PPA2 or Lu-AAZTA-PPA2.

### Ex Vivo Organ Distribution

FIG. 11 shows the ex vivo organ distribution of [⁶⁸Ga]Ga-DAZTA⁵-PPA2 in HEK293-SST2R positive (+) tumor bearing male SCID mice. The organs were extracted 1 h and 4 h post injection. Furthermore, tumor specificity was analyzed via blocking through administration of 100 μg Octreotide (TATE) 4 h post injection.

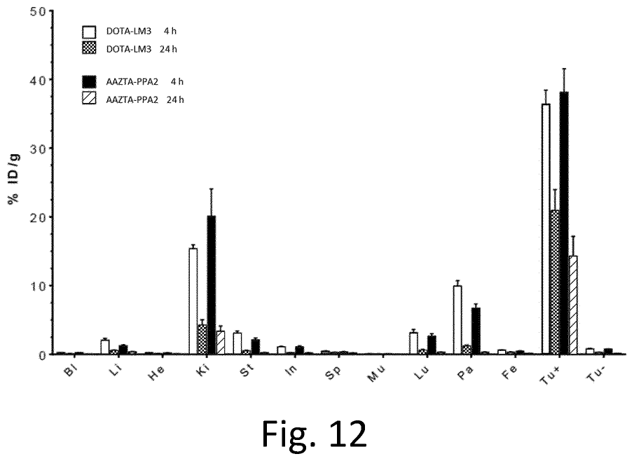

FIG. 12 depicts the ex vivo organ distribution of [¹¹¹In]In-AAZTA-PPA2 in HEK293-SST2R positive (+) and negative (−) tumor bearing male SCID mice in comparison to [¹¹¹In]In-DOTA-LM3. The organs were extracted 4 h and 24 h post injection.

1. Precursor DAZTA⁵-PPA2 for neuroendocrine theranostics having structure 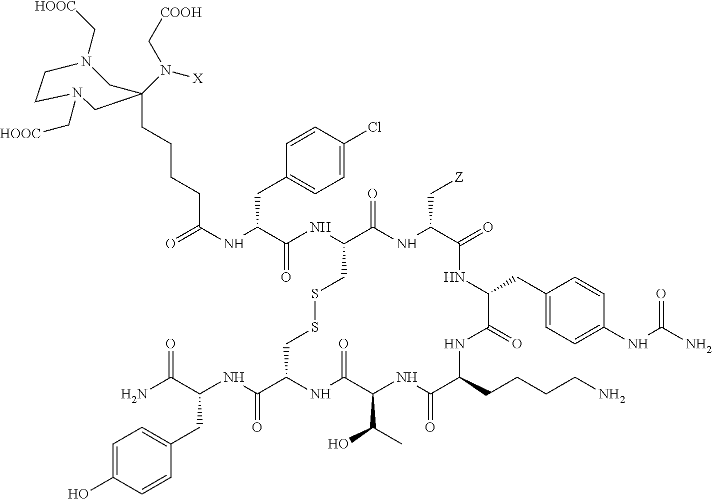
with 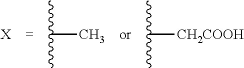
and 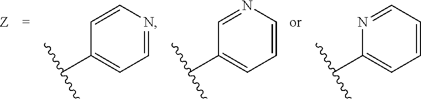
or a salt thereof.

2. Radiotracer ⁶⁸Ga-DAZTA⁵-PPA2 according to claim 1 consisting of precursor DAZTA⁵-PPA2 with 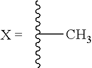
and therewith complexed radioisotope ⁶⁸Ga.

3. Radiotracer ¹⁷⁷Lu-DAZTA⁵-PPA2 according to claim 1 consisting of precursor DAZTA⁵-PPA2 with 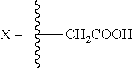
and therewith complexed radioisotope ¹⁷⁷Lu.

4. Radiopharmaceutical kit according to claim 1 comprising precursor DAZTA⁵-PPA2 with 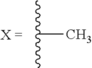
or a salt thereof.

5. Radiopharmaceutical kit according to claim 1 comprising precursor DAZTA⁵-PPA2 with 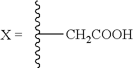
or a salt thereof.

6. Radiopharmaceutical kit according to claim 4 comprising a solvent selected from water, 0.45% aqueous NaCl solution, 0.9% aqueous NaCl solution, Ringer solution (Ringer lactate), 5% aqueous dextrose solution and aqueous alcohol solution.

7. Radiopharmaceutical kit according to claim 1 comprising
a first vial containing precursor DAZTA⁵-PPA2 with 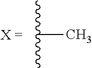
or a salt thereof, and
a second vial containing precursor DAZTA⁵-PPA2 with 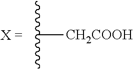
or a salt thereof.

8. Radiopharmaceutical kit according to claim 7 comprising one or two solvents selected independently of one another from water, 0.45% aqueous NaCl solution, 0.9% aqueous NaCl solution, Ringer solution (Ringer lactate), 5% aqueous dextrose solution and aqueous alcohol solution.

9. Use of the precursor of claim 1 for PET imaging, SPECT imaging or endoradiotherapy of somatostatin expressing tissue.

10. Use of the radiotracer of claim 2 for PET imaging, SPECT imaging or endoradiotherapy of somatostatin expressing tissue.

11. Use of the radiopharmaceutical kit of claim 6 for PET imaging, SPECT imaging or endoradiotherapy of somatostatin expressing tissue.
